(19) United States
(12) Patent Application Publication
(10) Pub. No.: US 2023/0347339 A1
(43) Pub. Date: Nov. 2, 2023

(54) DEVICE FOR DRIVING A CELL PROCESSING CHIP AND METHOD OF DRIVING A CELL PROCESSING CHIP

(71) Applicants: BEIJING BOE TECHNOLOGY DEVELOPMENT CO., LTD., Beijing (CN); BOE TECHNOLOGY GROUP CO., LTD., Beijing (CN)

(72) Inventors: Lin DENG, Beijing (CN); Da LI, Beijing (CN); Fan YANG, Beijing (CN); Xiangguo MA, Beijing (CN); Ding DING, Beijing (CN)

(21) Appl. No.: 17/637,334

(22) PCT Filed: Apr. 27, 2021
(86) PCT No.: PCT/CN2021/090300

§ 371 Date: Feb. 22, 2022

(51) Int. Cl. B01L 3/00

(52) U.S. Cl. CPC B01L 3/502715; B01L 3/50273; B01L 2300/0627

(57) ABSTRACT

The present application discloses a device for driving a cell processing chip and a method of driving a cell processing chip. The cell processing chip is configured to process cells. The device for driving the cell processing chip includes a bearing member configured to carry the cell processing chip, an accommodating member in fluid communication with the cell processing chip, a fluid driving member configured to drive flow of fluid in the device and in the cell processing chip, a signal generating and processing member configured to apply a signal to a fluid in the cell processing chip to generate a response signal associated with the fluid, and configured to issue a control instruction, and a power supply configured to supply power to the fluid driving member, and configured to apply a sorting signal to the cell processing chip in response to the control instruction.

### FIELD

The present disclosure relates to the field of biological detection, and more particularly to a device for driving a cell processing chip and a method of driving a cell processing chip.

### BACKGROUND

Microfluidic chip, also known as lab-on-a-chip, refers to the integration of basic operation units such as sample preparation, reaction, separation and detection involved in the fields of biology, chemistry and medicine into a chip with micron scale microchannels to automatically complete the whole process of reaction and analysis. The analysis and detection device based on microfluidic chip can have the following advantages: less sample consumption, fast analysis speed and very suitable for real-time and field analysis. Moreover, microfluidic chips can be designed as disposable products, which can eliminate complex liquid path systems such as cleaning and waste liquid treatment.

### SUMMARY

On the one hand, a device for driving a cell processing chip is provided. The cell processing chip is configured to process cells. The device comprises: a bearing member configured to carry the cell processing chip; an accommodating member in fluid communication with the cell processing chip and comprising a space for accommodating a sample or reagent; a fluid driving member configured to drive flow of fluid in the device and in the cell processing chip; a signal generating and processing member configured to apply a signal to a fluid in the cell processing chip to generate a response signal associated with the fluid, and configured to issue a control instruction in response to the response signal; and a power supply configured to supply power to the fluid driving member and the signal generating and processing member, and configured to apply a sorting signal to the cell processing chip in response to the control instruction.

In some embodiments, the signal generating and processing member comprises: a light source configured to provide an optical signal to the fluid in the cell processing chip through an optical transmission medium; an optical sensor configured to receive a response signal of the fluid through the optical transmission medium; and a processor configured to issue a control instruction based on a result of analyzing the response signal.

In some embodiments, the light source is connected to a first position through the optical transmission medium, the optical sensor is connected to a second position through the optical transmission medium, the first position and the second position are in proximity to the cell processing chip and are respectively located on both sides of a flow channel of the cell processing chip, and a connecting line between the first position and the second position passes through the flow channel.

In some embodiments, the device further comprises a rack, which is provided with a space for accommodating functional components; wherein the signal generating and processing member further comprises an optical transmission medium adapter plate detachably connected with the rack, and the optical transmission medium adapter plate is arranged between the light source and the first position or between the optical sensor and the second position. The optical transmission medium adapter plate comprises: an optical transmission medium section comprising a first end and a second end opposite to the first end, wherein the first end is arranged at the first position when the optical transmission medium adapter plate is arranged between the light source and the first position, the first end is arranged at the second position when the optical transmission medium adapter plate is arranged between the optical sensor and the second position, wherein the optical transmission medium at the first end is exposed, and the first end is fixed relative to a microfluidic chip when the optical transmission medium adapter plate is in the installation position; and wherein the second end is detachably connected to the light source when the first end is arranged at the first position, and the second end is detachably connected to the optical sensor when the first end is arranged at the second position.

In some embodiments, the light source is connected to a first optical transmission medium interface in the cell processing chip through the optical transmission medium, and the optical sensor is connected to a second optical transmission medium interface in the cell processing chip through the optical transmission medium, the first optical transmission medium interface and the second optical transmission medium interface are respectively connected to a transmitter and a receiver located in the cell processing chip and arranged opposite to each other on both sides of the flow channel of the cell processing chip.

In some embodiments, the device further comprises a rack, which is provided with a space for accommodating functional components; wherein the signal generating and processing member further comprises an optical transmission medium adapter plate detachably connected with the rack, and the optical transmission medium adapter plate is arranged between the light source and the first optical transmission medium interface or between the optical sensor and the second optical transmission medium interface. The optical transmission medium adapter plate comprises: an optical transmission medium section comprising a first end and a second end opposite to the first end; a first adapter arranged at the first end and configured to detachably connect the first end and the first optical transmission medium interface when the optical transmission medium adapter plate is arranged between the light source and the first optical transmission medium interface, and detachably connect the first end and the second optical transmission medium interface when the optical transmission medium adapter plate is arranged between the optical sensor and the second optical transmission medium interface; and wherein the second end is detachably connected to the light source when the optical transmission medium adapter plate is arranged between the light source and the first optical transmission medium interface, and the second end is detachably connected to the optical sensor when the optical transmission medium adapter plate is arranged between the optical sensor and the second optical transmission medium interface.

In some embodiments, the optical transmission medium adapter plate further comprises: a substrate detachably connected with the rack; an optical transmission medium sleeve covering at least part of the optical transmission medium section; and an adapter base connected with the optical transmission medium sleeve and the substrate, and the adapter base being provided with a through hole for the optical transmission medium to pass through.

In some embodiments, the adapter base is provided with a recess, and the optical transmission medium sleeve extends at the recess so that the intersection line of the optical transmission medium sleeve and the recess forms an arc.

In some embodiments, the fluid driving member comprises at least one air pump component, and each of the at least one air pump component comprises: an air pump inlet in fluid communication with an air pressure device outside the device; an air pump outlet configured to output high pressure gas; and an air pump controller configured to control flow of the air pump inlet and the air pump outlet.

In some embodiments, the accommodating member comprises at least one accommodating component, each of the at least one accommodating component comprises: a sample tube; an adapter arranged at one end of the sample tube and at least partially covering the sample tube; an air path connector arranged on the adapter and configured to be in fluid communication with the air pump outlet; and a liquid path connector arranged on the adapter and configured for liquid to enter and exit the sample tube.

In some embodiments, the at least one accommodating component comprises a first accommodating component, a second accommodating component and a third accommodating component, the cell processing chip comprises a first liquid inlet, a second liquid inlet and a liquid outlet, wherein liquid path connectors of the first accommodating component and the second accommodating component are in fluid communication with the first liquid inlet and the second liquid inlet respectively, and a liquid path connector of the third accommodating component is in fluid communication with the liquid outlet.

In some embodiments, the at least on air pump component comprises a first air pump component and a second air pump component, wherein the air path connectors of the first accommodating component and the second accommodating component are in fluid communication with the air pump outlets of the first air pump component and the second air pump component respectively.

In some embodiments, the optical transmission medium adapter plate further comprises substrate positioning magnets distributed at four corners of the substrate, and the rack comprises rack positioning magnets, wherein when the optical transmission medium adapter plate is in the installation position, the rack positioning magnets are magnetically combined with the optical substrate positioning magnets.

In some embodiments, the light source comprises an LED light source, the optical sensor comprises a PMT detector, and the optical transmission medium comprises an optical fiber or an optical waveguide.

In some embodiments, the bearing member comprises: a chip base provided with a groove; a chip fixing plate configured to be embedded in the groove in the installation state, and the chip fixing plate comprising a notch; wherein the cell processing chip is configured to be embedded in the notch in the installation state.

In some embodiments, the chip fixing plate comprises at least one fastening component, wherein when the at least one fastening component is in a fastening state, the at least one fastening component is configured to fasten the cell processing chip and the chip fixing plate to the chip base.

In some embodiments, the device further comprises: a top plate; and a bottom plate located on a side of the rack away from the top plate.

In some embodiments, the power supply comprises a first power supply, a second power supply and a third power supply that are mutually independent, the first power supply is configured to supply power to the fluid driving member, the second power supply is configured to supply power to the signal generating and processing member, and the third power supply is configured to apply a sorting signal to the cell processing chip in response to the control instruction.

On the other hand, a method of driving a cell processing chip using the device mentioned above is provided, wherein the cell processing chip comprises a droplet generation chip, the method comprises: fluidly communicating the accommodating member with the droplet generation chip and fluidly communicating the accommodating member with the fluid driving member, installing the droplet generation chip on the bearing member, adding an oil phase liquid and a cell suspension into the accommodating member; and supplying power to the fluid driving member using the power supply, so as to drive the fluid in the accommodating member into the droplet generation chip, thereby generating droplets wrapped in oil phase, and collecting the droplets wrapped in oil phase.

In some embodiments, after collecting the droplets wrapped in oil phase, the method further comprises: incubating the droplets wrapped in oil phase.

In some embodiments, the cell processing chip further comprises a droplet sorting chip; the device further comprises a signal generating and processing member configured to apply a signal to a fluid in the cell processing chip to generate a response signal associated with the fluid, and configured to issue a control instruction in response to the response signal; the power supply is further configured to supply power to the signal generating and processing member, and configured to apply a sorting signal to the cell processing chip in response to the control instruction; and wherein after incubating the droplets wrapped in oil phase, the method further comprises: fluidly communicating the accommodating member with the droplet sorting chip and fluidly communicating the accommodating member with the fluid driving member, coupling the signal generating and processing member with the droplet sorting chip, installing the droplet sorting chip on the bearing member, adding the incubated droplets wrapped in oil phase and oil phase liquid into the accommodating member; supplying power to the fluid driving member using the power supply, so as to drive the fluid in the accommodating member into the droplet sorting chip; and supplying power to the signal generating and processing member using the power supply to apply a signal to the fluid in the cell processing chip so as to generate a response signal associated with the fluid and issue a control instruction in response to the response signal, so that the power supply applies a sorting signal to the cell processing chip in response to the control instruction, thereby performing droplet sorting.

### DETAILED DESCRIPTION OF THE DRAWINGS

In order to explain the technical scheme in the embodiments of the present disclosure more clearly, the accompanying drawings to be used in the description of the embodiments will be briefly introduced below. Obviously, the drawings described below are only some embodiments of the present disclosure.

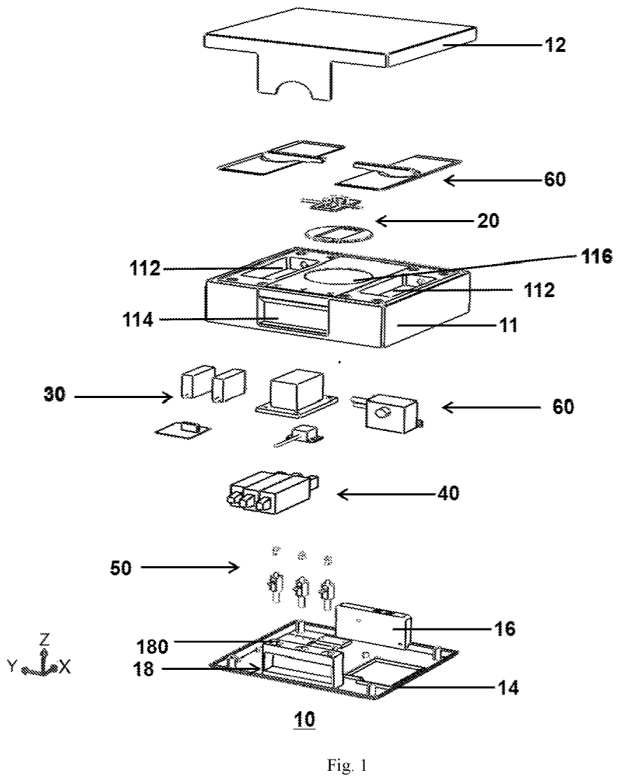

FIG. 1 shows a component exploded view of a device for driving a cell processing chip according to some embodiments of the present disclosure;

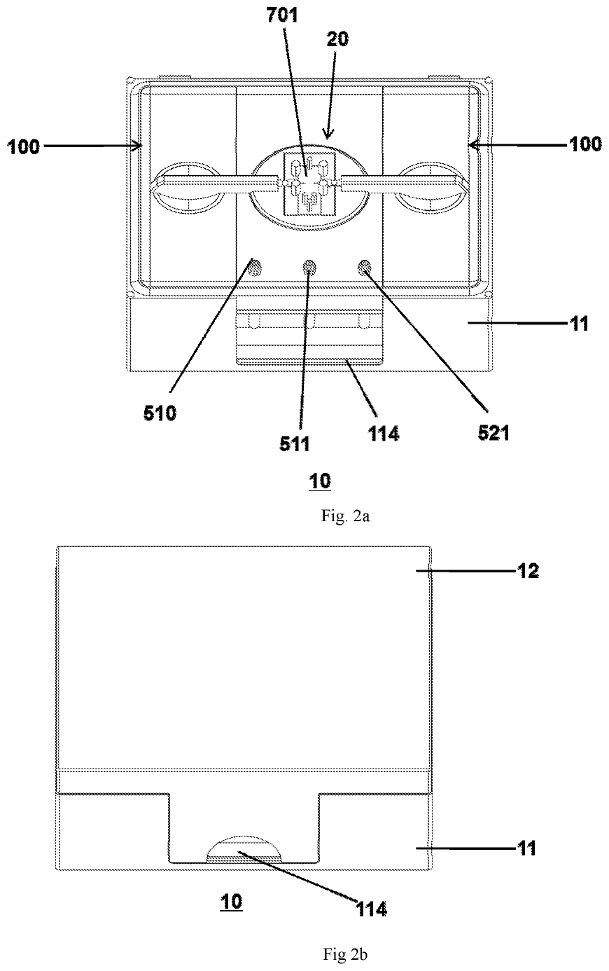

FIG. 2a shows an appearance diagram of a device for driving a cell processing chip according to some embodiments of the present disclosure, and FIG. 2b shows an appearance diagram of the device of FIG. 2a in the case of including a top plate;

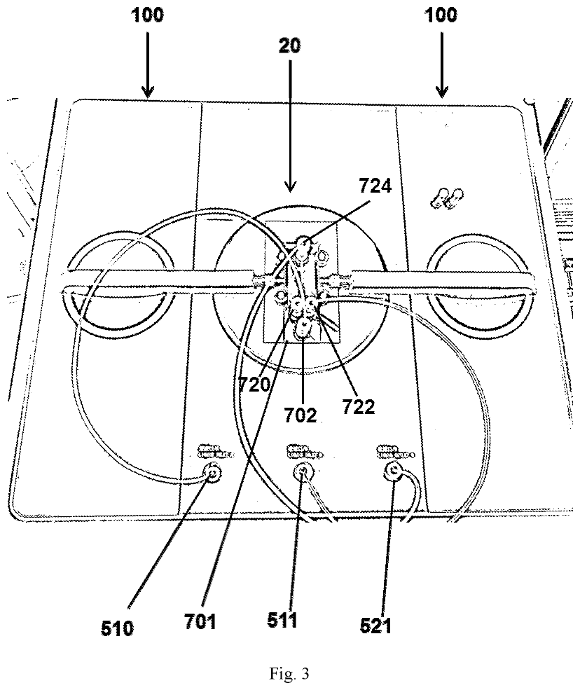

FIG. 3 shows an appearance diagram of a partial structure of a device for driving a cell processing chip according to some embodiments of the present disclosure;

FIGS. 4-6 schematically show an internal structure diagram of a device according to some embodiments of the present disclosure from different perspectives;

FIG. 7 schematically shows a structural diagram of an accommodating member according to some embodiments of the present disclosure from different perspectives;

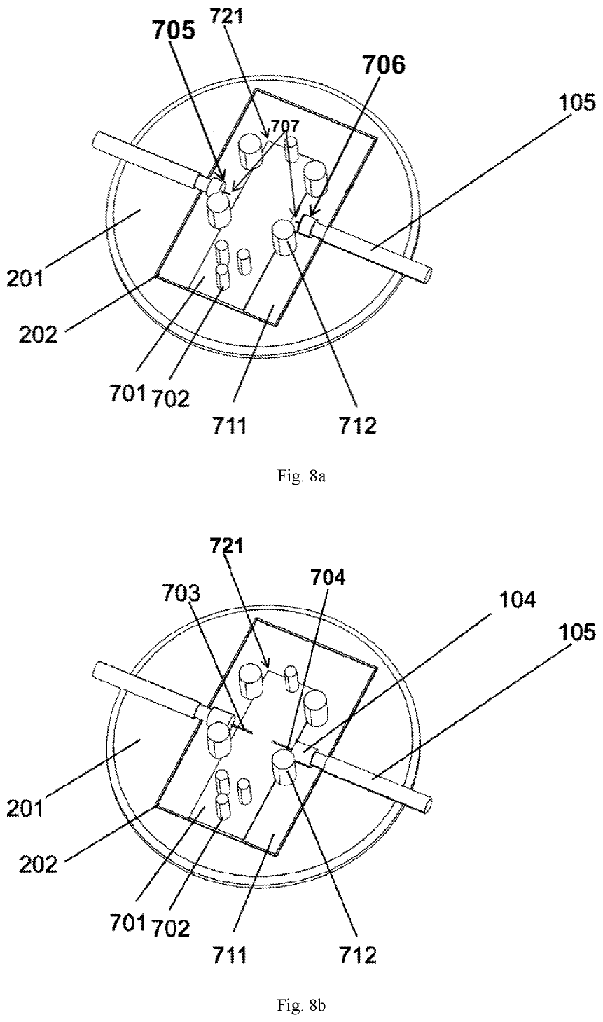

FIGS. 8a-8b schematically show a partial structural diagram of a device for driving a cell processing chip according to some embodiments of the present disclosure;

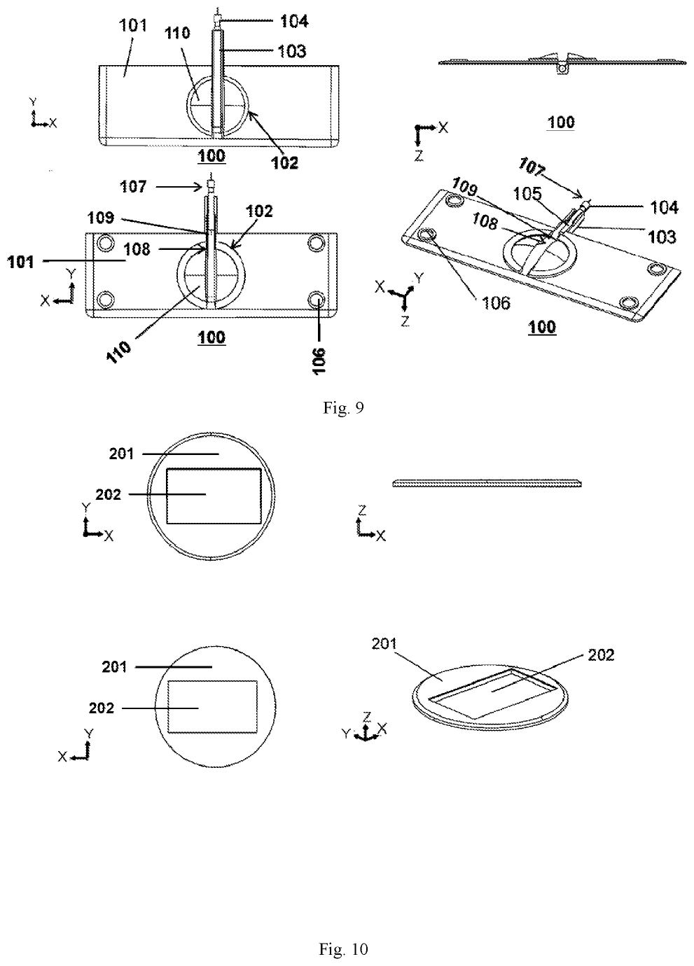

FIG. 9 schematically shows a structural diagram of an optical transmission medium adapter plate according to some embodiments of the present disclosure from different perspectives;

FIG. 10 schematically shows a partial structural diagram of a bearing member according to some embodiments of the present disclosure from different perspectives;

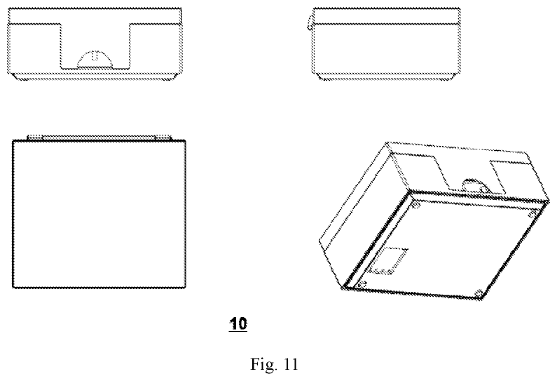

FIG. 11 schematically shows a view of a device for driving a cell processing chip according to some embodiments of the present disclosure from different perspectives; and

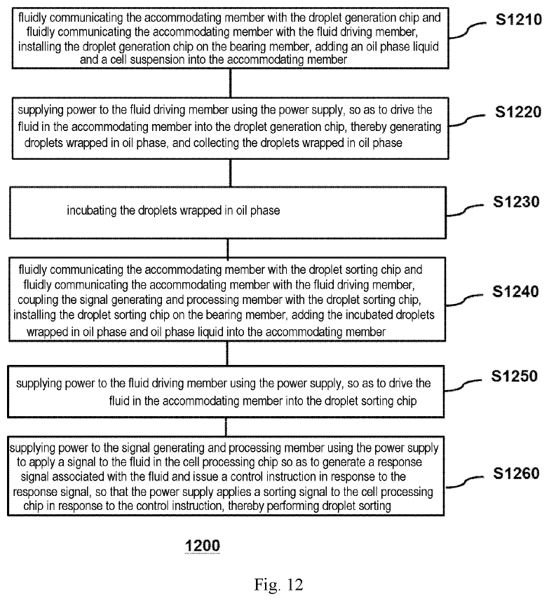

FIG. 12 schematically shows a flowchart of a method of driving a cell processing chip according to some embodiments of the present disclosure.

### EMBODIMENTS

In order to make the purpose, technical scheme and advantages of the embodiments of the present disclosure more clear, the technical scheme of the embodiments of the present disclosure will be further described in detail below in combination with the accompanying drawings.

It should be understood that although the terms first, second, third, etc. can be used herein to describe various elements, components and/or parts, these elements, components and/or parts should not be limited by these terms. These terms are used only to distinguish one element, component or part from another. Therefore, the first element, component or part discussed below may be referred to as a second element, component or part without departing from the teaching of the present disclosure.

The terms used herein are for the purpose of describing specific embodiments only and are not intended to limit the present disclosure. As used herein, the singular forms “a”, “one” and “the” are intended to include the plural unless otherwise indicated clearly in the context. It will be further understood that the terms “comprise” and/or “include”, when used in this specification, specify the presence of the described features, entirety, steps, operations, elements and/or components, while not excluding presence of one or more other features, entirety, steps, operations, elements, components and/or groups thereof or adding one or more other features, entirety, steps, operations, elements, components and/or groups thereof. As used herein, the term “and/or” includes any and all combinations of one or more of the associated listed items. As used herein, the term “A or B” is used to refer to at least one of item A and item B without contradiction.

Cells are the basic structural and functional units of organisms. The inventor of the present application found that due to the heterogeneity among various cell populations, the mean value obtained through the analysis of cell populations masks the differences between individual cells, cannot characterize the stochastic nature of gene expression, and cannot reflect the real situation. The inventor of the application found that with the proposal of the concept of precision medicine, cell population analysis has developed to single cell analysis. Single cell analysis is mainly divided into four parts: single cell sorting, sample pretreatment, reaction, detection and analysis. The key of single cell analysis is to separate single cells from highly heterogeneous biological samples. The inventor of the present application found that, single cell sorting methods in related technologies are mainly divided into two categories: one is dependent on fluorescence activated cell sorting (FACS) for automatic operation, and the purchase and maintenance of the instrument are expensive; the second is the manual single cell sorting method, which depends on the skills and proficiency of operators, and also requires large and medium-sized instruments such as micropipette platform and optical tweezers. The inventor of the application further found that the sorting method in the related technology is expensive, requires high personnel skills, and the required instruments and equipment are limited by the site. In addition, the single cell sorting process is easily polluted by aerosols and microorganisms floating in the environment, which is usually difficult to be completely removed in the subsequent detection links. Therefore, the research and development of small and portable single cell sorting equipment is of great practical significance to reduce the use cost, reduce the dependence on the skills of operators and reduce cross pollution.

An embodiment of the present application provides a device for driving a cell processing chip. FIG. 1 shows a component exploded view of a device for driving a cell processing chip according to some embodiments of the present disclosure. FIG. 2a shows an appearance diagram of the device 10 for driving a cell processing chip 701 according to some embodiments of the present disclosure, and FIG. 2b shows an appearance diagram of the device 10 of FIG. 2a in the case of including a top plate. FIG. 3 shows an appearance diagram of a partial structure of the device 10 for driving the cell processing chip 701 according to some embodiments of the present disclosure. Referring to FIGS. 1 to 3, the cell processing chip 701 is configured to process cells. The device 10 includes: a bearing member 20 configured to carry a cell processing chip 701; an accommodating member 50 in fluid communication with the cell processing chip 701 and including a space for accommodating a sample or reagent; a fluid driving member 30 configured to drive the flow of fluid in the device 10 and in the cell processing chip 701; a signal generating and processing member 60 configured to apply a signal to the fluid in the cell processing chip 701 to generate a response signal associated with the fluid, and configured to issue a control instruction in response to the response signal; and a power supply 40 configured to supply power to the fluid driving member 30 and the signal generating and processing member 60 and configured to apply a sorting signal to the cell processing chip 701 in response to the control instruction.

It should be understood that in the description of the present disclosure, the term “fluid communication” is used to refer to a channel isolated from the external environment between different elements, which can be used for fluid flow or transmission. For example, fluid communication between cavity A and cavity B can be achieved by a plastic pipe connecting cavity A and cavity B.

An embodiment of the present application provides a device for driving a cell processing chip, which comprises several functional members to realize the effective driving of the cell processing chip, so as to realize the processing of cells. The device has compact and simple structure, and can realize miniaturization and portability, so as to effectively reduce the overall size of the instrument and simplify the operation process. At the same time, it can automate the operation, reduce the dependence on the skills of operators, eliminate cross pollution, and reduce the damage or impact on cells in the process of cell treatment. By arranging the signal generating and processing member 60, a signal can be applied to the fluid in the cell processing chip 701 to generate a response signal associated with the fluid, and a control instruction can be issued in response to the response signal, so as to realize real-time sensing and feedback of the fluid situation in the chip, which is conducive to the integration of multiple functions in the same chip, and improves the effect of cell treatment.

FIGS. 4-6 schematically show an internal structure diagram of a device according to some embodiments of the present disclosure from different perspectives. Referring to FIGS. 4-6, in some embodiments, the fluid driving member 30 may comprise at least one air pump component 310, 316, each of the at least one air pump component 310, 316 comprising: an air pump inlet 303 (or 313) in fluid communication with an air pressure device outside the device 10 (e.g., an air compressor or a high-pressure gas cylinder, not shown); an air pump outlet 302 (or 312) configured to output high-pressure gas; and an air pump controller 301 (or 311) configured to control the flow of the air pump inlet 303 (or 313) and the air pump outlet 302 (or 312). By arranging the air pump components, the liquid flow in the device 10 and the cell processing chip 701 can be driven by air pressure, so as to realize efficient and accurate driving.

In some embodiments, the fluid driving member 30 may further comprise an air pump power board 320 connected to the power supply 40 and configured to drive and control the air pump controller 301 (or 311).

In some embodiments, the accommodating member 50 may comprise at least one accommodating component. FIG. 7 schematically shows a structural diagram of the accommodating components 510, 511, 521 according to some embodiments of the present disclosure from different perspectives. Referring to FIGS. 1-7, each of the at least one accommodating component 510, 511, 521 comprises: a sample tube 501; an adapter 502 arranged at one end of the sample tube 501 and at least partly covering the sample tube 501; an air path connector 503 arranged on the adapter 502 and configured to be in fluid communication with the air pump outlet 302 (or 312); and a liquid path connector 504 arranged on the adapter 502 and configured for liquid to enter and exit the sample tube 501. By arranging the accommodating components, the sample tube 501 can be in fluid communication with the air path connector 503 and the liquid path connector 504, so that the liquid flow in the device 10 and the cell processing chip 701 can be driven by the air pressure from the air pump component, and efficient and accurate driving can be realized.

In some embodiments, referring to FIGS. 2a-4, the at least one accommodating component comprises a first accommodating component 510, a second accommodating component 511 and a third accommodating component 521. The cell processing chip 701 comprises a first liquid inlet 702, a second liquid inlet 722 and a liquid outlet 724, wherein liquid path connectors of the first accommodating component 510 and the second accommodating component 511 are in fluid communication with the first liquid inlet 702 and the second liquid inlet 722 respectively, and a liquid path connector of the third accommodating component 521 is in fluid communication with the liquid outlet 724. By fluidly communicating a plurality of accommodating components with the first liquid inlet 702, the second liquid inlet 722 and the liquid outlet 724 of the cell processing chip 701, the liquid samples or reagents in different accommodating components can be driven into the fluid driving component to avoid cross contamination, and the liquid treated by the cell processing chip 701 can be collected. For example, as shown in FIG. 3, the first liquid inlet 702, the second liquid inlet 722, a third liquid inlet 720 and the liquid outlet 724 can be realized through holes arranged on the upper surface of the cell processing chip 701, and the fluid communication can be realized through a plastic hose. For example, the cell processing chip 701 may further comprise a fixing component for fixing the plastic hose to the first liquid inlet 702, the second liquid inlet 722, the third liquid inlet 720 and the liquid outlet 724.

In some embodiments, the cell processing chip 701 may further comprise a third liquid inlet 720. In this way, it is convenient for function expansion. In addition, in some embodiments, the liquid path connectors of the first accommodating component 510, the second accommodating component 511 and the third accommodating component 521 of the device 10 may be in fluid communication with the first liquid inlet 702, the second liquid inlet 722 and the third liquid inlet 720 of the cell processing chip, respectively, while the liquid outlet 724 of the cell processing chip is in fluid communication with other containers.

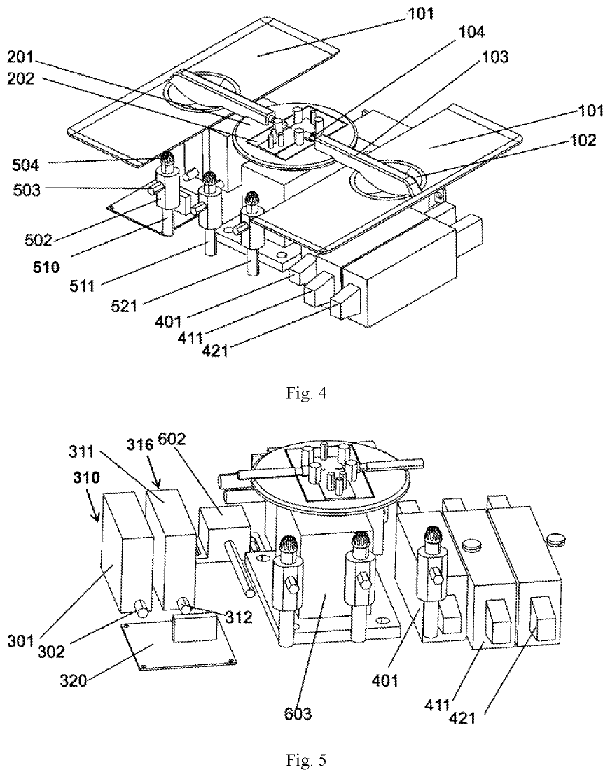

In some embodiments, referring to FIG. 5, the at least one air pump component comprises a first air pump component 310 and a second air pump component 316, wherein the air path connectors of the first accommodating component 510 and the second accommodating component 511 are in fluid communication with the air pump outlets of the first air pump component 310 and the second air pump component 316. In this way, the first air pump component 310 and the second air pump component 316 can drive the fluid of the first accommodating component 510 and the second accommodating component 511 respectively.

In some embodiments, the same air pump component can also drive two or more accommodating components at the same time, that is, the air pump outlet of the same air pump component is in fluid communication with the air path connectors of two or more accommodating components at the same time.

FIGS. 8a-8b schematically show a partial structural diagram of a device for driving a cell processing chip 701 according to some embodiments of the present disclosure. In some embodiments, referring to FIGS. 1, 5-6, and 8a-8b, the signal generating and processing member 60 comprises: a light source 601 configured to provide an optical signal to a fluid in the cell processing chip 701 through an optical transmission medium; an optical sensor 603 configured to receive a response signal of the fluid through the optical transmission medium; and a processor (not shown) configured to issue a control instruction based on the result of analyzing the response signal.

In some embodiments, the light source 601 is connected to a first position 705 through the optical transmission medium. The optical sensor 603 is connected to a second position 706 through the optical transmission medium. The first position 705 and the second position 706 are in proximity to the cell processing chip 701 and are respectively located on both sides of a flow channel of the cell processing chip 701, and a connecting line between the first position 705 and the second position 706 passes through the flow channel. The “proximity” here is used to indicate a distance within which the light source 601 can directly transmit an optical signal to the flow channel through the optical transmission medium, and the optical sensor 603 can directly receive a response signal from the flow channel through the optical transmission medium. The present disclosure does not limit the specific positions of the first position and the second position, as long as the optical signal can be transmitted and received normally. In this way, the light source 601 can directly transmit an optical signal to the flow channel through the optical transmission medium, and the optical sensor 603 can directly receive a response signal from the flow channel through the optical transmission medium without other optical components, which simplifies the structure. The optical transmission medium connected to the light source 601 and the optical transmission medium connected to the optical sensor 603 can transmit and receive signals without even entering the cell processing chip 701 (for example, located at a distance from the cell processing chip 701), which simplifies the structure.

In this case, referring to FIGS. 1-4, 6, 8a and 9, the device 10 may further comprise a rack 11, which is provided with a space for accommodating functional components. The signal generating and processing member 60 may further comprise an optical transmission medium adapter plate 100 detachably connected with the rack 11, and the optical transmission medium adapter plate 100 is arranged between the light source 601 and the first position 705 or between the optical sensor 603 and the second position 706. The optical transmission medium adapter plate 100 comprises: an optical transmission medium section 105 comprising a first end 107 and a second end 108 opposite to the first end 107. The first end 107 is arranged at the first position 705 when the optical transmission medium adapter plate 100 is arranged between the light source 601 and the first position 705, and the first end 107 is arranged at the second position 706 when the optical transmission medium adapter plate 100 is arranged between the optical sensor 603 and the second position 706, wherein the optical transmission medium 707 at the first end 107 is exposed and the first end 107 is fixed relative to the microfluidic chip 701 when the optical transmission medium adapter plate 100 is in the installation position; and wherein the second end 108 is detachably connected to the light source 601 when the first end 107 is arranged at the first position 705, and the second end 108 is detachably connected to the optical sensor 603 when the first end 107 is arranged at the second position 706.

In this way, by arranging an optical transmission medium adapter plate between the light source and the first optical transmission medium interface or between the optical sensor and the second optical transmission medium interface, the first end 107 being fixed relative to the microfluidic chip 701 when the optical transmission medium adapter plate is in the installation position, the substrate being detachably connected with the rack and the optical transmission medium adapter plate comprising an optical transmission medium section, it ensures that the optical transmission medium plate can be disassembled from the rack while ensuring normal transmitting and receiving of signals, so as to provide a transitional section between the light source and the first optical transmission medium interface or between the optical sensor and the second optical transmission medium interface, which facilitates installation and disassembly of the cell processing chip 701 and replacement of other components, and facilitates inspection of the operation status of the device.

In some embodiment, the light source 601 is connected to a first optical transmission medium interface 703 in the cell processing chip 701 through the optical transmission medium, and the optical sensor 603 is connected to a second optical transmission medium interface 704 in the cell processing chip 701 through the optical transmission medium. The first optical transmission medium interface 703 and the second optical transmission medium interface 704 are respectively connected to a transmitter and a receiver (not shown) located in the cell processing chip 701 and arranged opposite to each other on both sides of the flow channel (not shown) of the cell processing chip 701. For example, the transmitter built in the cell processing chip 701 (such as an optical transmission medium or optical element embedded in the chip) to which the first optical transmission medium interface 703 is connected is used to transmit the optical signal sent by the light source, and the receiver built in the cell processing chip 701 (such as an optical transmission medium or optical element embedded in the chip) to which the second optical transmission medium interface 704 is connected is used to collect an optical signal (response signal) transmitted or reflected by the fluid in the cell processing chip 701.

In this way, the light emitted by the light source can be transmitted to the fluid in the cell processing chip 701 and the optical signal (response signal) transmitted or reflected by the fluid can be collected by using the transmitter and receiver located in the cell processing chip 701 and arranged opposite to each other on both sides of the flow channel (not shown), which improves the quality of the signal and the efficiency of collection.

In this case, referring to FIGS. 1-4, 6, 8b and 9, the device 10 may further comprise a rack 11, which is provided with spaces 112 and 116 for accommodating functional components. The signal generating and processing member 60 may further comprise an optical transmission medium adapter plate 100 detachably connected with the rack 11, and the optical transmission medium adapter plate 100 is arranged between the light source 601 and the first optical transmission medium interface 703 or between the optical sensor 603 and the second optical transmission medium interface 704. The optical transmission medium adapter plate 100 comprises: an optical transmission medium section 105 comprising a first end 107 and a second end 108 opposite to the first end 107; a first adapter 104 arranged at the first end 107 and configured to detachably connect the first end 107 and the first optical transmission medium interface 703 when the optical transmission medium adapter plate 100 is arranged between the light source 601 and the first optical transmission medium interface 703, and detachably connect the first end 107 and the second optical transmission medium interface 704 when the optical transmission medium adapter plate 100 is arranged between the optical sensor 603 and the second optical transmission medium interface 704; and wherein the second end 108 is detachably connected to the light source 601 (for example, through a second connector 109) when the optical transmission medium adapter plate 100 is arranged between the light source 601 and the first optical transmission medium interface 703, and the second end 108 is detachably connected to the optical sensor 603 (for example, through the second connector 109) when the optical transmission medium adapter plate 100 is arranged between the optical sensor 603 and the second optical transmission medium interface 704.

By arranging an optical transmission medium adapter plate between the light source and the first optical transmission medium interface or between the optical sensor and the second optical transmission medium interface, the substrate being detachably connected with the rack, and the optical transmission medium adapter plate comprising an optical transmission medium section, the optical transmission medium plate can be detachable from the rack while ensuring normal transmitting and receiving of signals, so as to provide a transitional section between the light source and the first optical transmission medium interface or between the optical sensor and the second optical transmission medium interface, which facilitates installation and disassembly of the cell processing chip 701 and replacement of other components, and facilitates inspection of the operation status of the device.

In some embodiments, the optical transmission medium adapter plate 100 further comprises: a substrate 101 detachably connected with the rack 11; an optical transmission medium sleeve 103 covering at least part of the optical transmission medium section 105; and an adapter base 102 connected (e.g., fixed connection) with the optical transmission medium sleeve 103 and the substrate 101, and the adapter base 102 being provided with a through hole (not shown) for the optical transmission medium to pass through.

By arranging an optical transmission medium sleeve 103 covering at least part of the optical transmission medium section 105, the optical transmission medium section 105 can be well protected, fixed and shielded. At the same time, the optical transmission medium sleeve 103 provides a larger operation area, which facilitates the operator to apply force to install or disassemble the optical transmission medium adapter plate 100, and improves the operation experience.

In some embodiments, the adapter base 102 is provided with a recess 110, and the optical transmission medium sleeve 103 extends at the recess 110 so that the intersection line of the optical transmission medium sleeve 103 and the recess 110 forms an arc. In this way, the adapter base 102 is provided with a recess 110 at the position connected with the optical transmission medium sleeve 103, which increases the extension area of the optical transmission medium sleeve 103 and further increases the operation area, so as to increase the area available for force application and improve the operation experience.

In some embodiments, the optical transmission medium adapter plate 100 further comprises substrate positioning magnets 106 distributed at four corners of the substrate 101, and the rack 11 comprises rack positioning magnets (not shown), wherein when the optical transmission medium adapter plate 100 is in the installation position, the rack positioning magnets 106 are magnetically combined with the optical substrate positioning magnets. In this way, the optical transmission medium adapter plate 100 in the installation position is combined with the rack 11 by magnetic force to facilitate the installation and disassembly of the optical transmission medium adapter plate 100 from the rack 11.

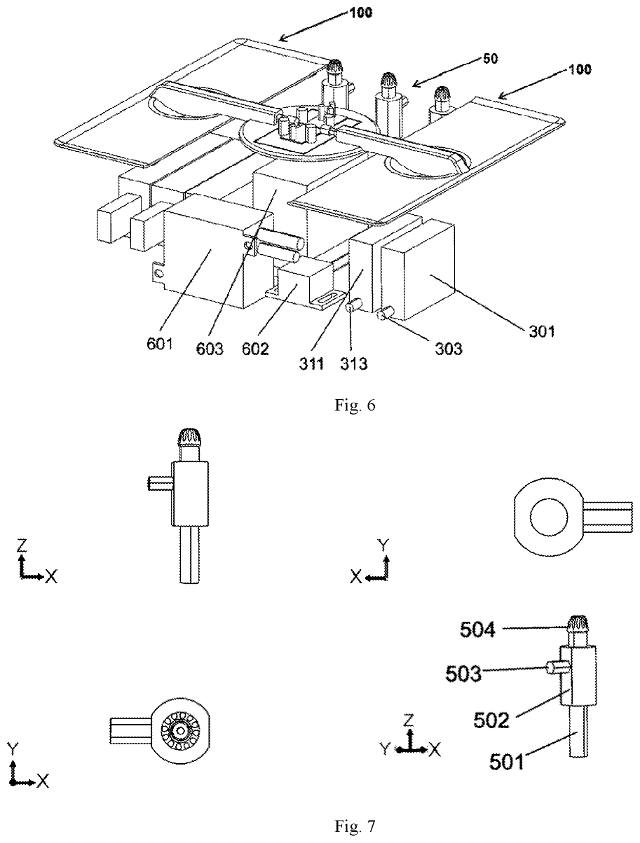

In some embodiments, the light source 601 comprises an LED light source (e.g., an LED excitation light source generator), the optical sensor 603 comprises a photomultiplier tube (PMT) detector, and the optical transmission medium 105 comprises an optical fiber or an optical waveguide. By using the PMT detector, the optical signals reflected or transmitted by the fluid can be effectively collected and analyzed, and excellent sensitivity and response speed can be realized. In some embodiments, as shown in FIG. 6, the signal generating and processing member 60 may further comprise a third adapter 602, such as an LED excitation light source optical fiber head, which is configured to convert the light emitted by the LED light source into a signal transmitted in the optical fiber. In this way, the free space optical path system can be simplified into an optical fiber optical path system, which simplifies the structure and reduces the cost.

FIG. 10 schematically shows a partial structural diagram of a bearing member 20 according to some embodiments of the present disclosure from different perspectives. In some embodiments, referring to FIGS. 1-6, 8a-8b, and 10, the bearing member 20 comprises: a chip base 201 provided with a groove 202; a chip fixing plate 711 configured to be embedded in the groove 202 in the installation state, and the chip fixing plate 711 comprising a notch 721; wherein the cell processing chip 701 is configured to be embedded in the notch 721 in the installation state. By arranging the groove 202 and the chip fixing plate 711, the cell processing chip 701 can be well fixed and positioned in the working state, and it is convenient to install and disassemble the cell processing chip 701.

In some embodiments, the chip fixing plate 711 comprises at least one fastening component 712 (for example, bolts), wherein when the at least one fastening component 712 is in a fastening state, the at least one fastening component 712 is configured to fasten the cell processing chip 701 and the chip fixing plate 711 to the chip base 201. In some embodiments, the orthographic projection of the cell processing chip 701 on the chip base 201 does not overlap with the orthographic projection of the chip fixing plate 711 on the chip base 201. In this way, the cell processing chip 701 in the working state can be well fixed and positioned.

In some embodiments, referring to FIGS. 1-2b, the device 10 further comprises: a top plate 12; and a bottom plate 14 located on a side of the rack 11 away from the top plate 12. The top plate 12, the bottom plate 14 and the rack 11 together form at least part of the structural member of the device 10, which can protect, locate and support various functional components. The material of the structural member can be resin, metal, etc., which is not limited in the present disclosure. In some embodiments, the top plate 12 may be at least partially optically transparent, thereby facilitating the observation of the operation inside the device.

In some embodiments, referring to FIGS. 1-2b, an opening 114 may be provided at one side of the rack 11, and the size of the opening 114 may be similar to the size of at least one accommodating component 510, 511 and 521, so as to facilitate the installation and disassembly of at least one accommodating components 510, 511 and 521 of the accommodating member 50.

In some embodiments, referring to FIG. 1, the device 10 may further comprise a first positioning plate 16. The first positioning plate 16 can be connected with the bottom plate 14, and the plane of the first positioning plate 16 can be approximately perpendicular to the plane of the bottom plate 14. The first positioning plate 16 can position the rack 11.

In some embodiments, referring to FIG. 1, the device 10 may further comprise a second positioning plate 18 opposite the first positioning plate 16. The second positioning plate 18 can be connected with the bottom plate 14, and the plane of the second positioning plate 18 can be approximately perpendicular to the plane of the bottom plate 14. The second positioning plate 18 can position the rack 11. The second positioning plate 18 may be provided with at least one through hole 180, and the axis of the at least one through hole 180 is perpendicular to the plane of the base plate 14. In this way, the accommodating components 510, 511 and 521 can be inserted into the at least one through hole 180 to fix and position the accommodating components 510, 511 and 521.

In some embodiments, referring to FIGS. 1 to 10, the optical sensor 603 is located between the fluid driving member 30 and the power supply 40, and the fluid driving member 30 and the power supply 40 are located on the left and right sides of the optical sensor 603. The optical sensor 603 is located between the bearing member 20 and the base plate 14, and the bearing member 20 is located directly above the optical sensor 603. The top plate 12 is located on a side of the bearing member 20 away from the light sensor 603. The light sensor 603 is located between the light source 601 and the accommodating member 50, and the light source 601 and the accommodating member 50 are located on the front and rear sides of the light sensor 603. The signal generating and processing member 60 comprises two optical transmission medium adapter plates 100 left and right, the two optical transmission medium adapter plates 100 are respectively located on the left and right sides of the bearing member 20. The two optical transmission medium adapter plates 100 are located directly above the fluid driving member 30 and the power supply 40, respectively. The rack 11 is provided with spaces 112 and 116 for accommodating functional components, the fluid driving member 30 and the power supply 40 can be accommodated in the space 112 of the rack 11 respectively, and the optical sensor 603 can be accommodated in the space 116 of the rack 11. One side of the rack 11 can be provided with an opening 114, the second positioning plate 18 can be provided with at least one through hole 180, the accommodating components 510, 511 and 521 can be inserted into the at least one through hole 180, and the second positioning plate 18 can be accommodated in the opening 114.

In some embodiments, referring to FIGS. 1, 4-5, the power supply 40 comprises a first power supply 401, a second power supply 411 and a third power supply 421 that are mutually independent, the first power supply 401 is configured to supply power to the fluid driving member 30, the second power supply 411 is configured to supply power to the signal generating and processing member 60, and the third power supply 421 is configured to apply a sorting signal to the cell processing chip 701 in response to a control instruction. For example, the first power supply 401 provides a voltage of 24 V, the second power supply 411 provides a voltage of 36 V, and the third power supply 421 provides a DC high voltage (e.g., 800 V-1000 V). In this way, different power supplies can be used to power different members, so as to realize a variety of functions.

FIG. 11 schematically shows a view of a device 10 for driving a cell processing chip 701 according to some embodiments of the present disclosure from different perspectives.

In some embodiments, the cell processing chip 701 can be a droplet generation chip, and the device 10 can realize the function of droplet generation in cooperation with the cell processing chip 701, that is, the device 10 is a droplet generation device. Specifically, by using microfluidic chip and droplet generation technology (the specific method can be described below), the droplet generation device uses a micro flow pump (at least one air pump component 310, 316) to control the mixing process of cell samples, oil phase liquid and biochemical reagent solution accommodated in the accommodating components 510, 511 and 521, encapsulates a single cell and subsequent reaction reagent in droplets, forms a micro reactor for biochemical reaction of a single cell by surfactant stabilization, and also constitutes a droplet carrier for cell sorting. For example, the continuous phase can be oil phase and the dispersed phase can be water phase.

In some embodiments, the cell processing chip 701 can be a cell sorting chip, and the device 10 can realize the function of cell sorting in cooperation with the cell processing chip 701, that is, the device 10 is a cell sorting device. Specifically, the cell sorting device uses a micro flow pump (at least one air pump component 310, 316) to control the mixing process of oil phase wrapped droplets and oil phase liquid accommodated in the accommodating components 510, 511 and 521, uses the microfluidic chip as the cell sorting chip, uses the light source to generate an optical signal to irradiate the fluid, and uses the optical sensor 603 to collect and analyze the response signal of the fluid, so as to generate a sorting signal to control the discharge of the high-voltage electrode (not shown) in the chip, and the dielectric power is used to sort single-cell droplets. In this way, the liquid flow path and its pump valve system can be effectively simplified. The detection object of cell sorting device is a single droplet. The size can be controlled from a few microns to tens of microns through liquid flow control. Therefore, the free space optical path system can be simplified into an optical fiber optical path system. In addition, the sorting method (the specific method can be described below) adopts the dielectrophoresis sorting method, and the high-voltage electric field is applied to the droplet surface, which effectively improves the cell survival rate. Compared with the high-pressure jet module used in large FACS devices in related technologies which has large size and obvious damage to cells, in the cell sorting device of the present disclosure, the whole preparation and sorting process is mild and protected by droplets, which effectively improves the survival rate of cells.

The sorting device provided by the embodiments of the present disclosure can realize single cell sorting. The device specifically relates to a control and detection device for a single cell sorting microfluidic chip for droplet generation, single cell encapsulation and fluorescence activation. The device can realize the operation of cell labeling, typing and sorting (for example, typing for circulating tumor cells, rare cells and specific cells in peripheral blood samples, etc.), thus providing a new choice for hot medical fields such as single cell analysis, early cancer diagnosis and concomitant diagnosis.

The embodiment of the present disclosure also provides a method of driving a cell processing chip using the aforementioned device. FIG. 12 schematically shows a flowchart of a method of driving a cell processing chip according to some embodiments of the present disclosure. In some embodiments, the cell processing chip comprises a droplet generation chip configured to wrap the droplets in oil phase. The method 1200 comprises the steps of S1210-S1220:

S1210: fluidly communicating the accommodating member with the droplet generation chip and fluidly communicating the accommodating member with the fluid driving member, installing the droplet generation chip on the bearing member, adding an oil phase liquid and a cell suspension into the accommodating member.

For example, the droplet generation chip is inserted into the chip fixing plate 711 and the fastening member 712 is tightened. The first liquid inlet 702 is fluidly communicated with the liquid path connector of the accommodating component 510 through the plastic hose, the second liquid inlet 722 is fluidly communicated with the liquid path connector of the accommodating component 511 through the plastic hose, and the liquid outlet 724 is fluidly communicated with the liquid path connector of the accommodating component 521 through the plastic hose. The air path connector of the accommodating component 510 is fluidly communicated with the air pump outlet 302 of the air pump component 310, the air path connector of the accommodating component 511 is fluidly communicated with the air pump outlet 312 of the air pump component 320, and the air pump inlets 303 and 313 are connected to the external air compressor or high-pressure gas cylinder. The chip fixing plate 711 connected with various connectors is inserted into the groove 202 of the chip base 201. An oil phase liquid (droplet generation oil) is added to the sample tube of the accommodating component 510 and a cell suspension is added to the sample tube of the accommodating component 511.

S1220: supplying power to the fluid driving member using the power supply, so as to drive the fluid in the accommodating member into the droplet generation chip, thereby generating droplets wrapped in oil phase, and collecting the droplets wrapped in oil phase.

For example, the air compressor is started (or the switch of the high-pressure gas cylinder is turned on), the power supply is used to power the air pump controller, and the ratio of air pressure for driving the sample tube of the accommodating component 510 and the sample tube of the accommodating component 511 is adjusted to control the liquid flow rate. The air pressure for driving the sample tube of the accommodating component 510 is increased firstly, so that oil phase liquid (droplet generation oil) first fills the droplet generation chip, and then the cell suspension is injected into the droplet generation chip. The droplets are collected for 2 minutes and discarded as waste liquid. After that, the droplets are collected and entered the sample tube of the third accommodating component 521 until the required amount.

In some embodiments, it is also necessary to add biochemical reagents to the droplet generation chip. In some embodiments, the biochemical reagents and the cell suspension may be pre proportioned and added to the sample tube of the accommodating component 511, and the waste liquid or collection liquid enters the sample tube of the accommodating component 521.

In other embodiments, the droplet generation chip may further comprise a third liquid inlet 720. The first liquid inlet 702 is fluidly communicated with the liquid path connector of the accommodating component 510 through a plastic hose, and the first liquid inlet 702 is used for oil phase liquid (droplet generation oil) to enter. The second liquid inlet 722 is fluidly communicated with the liquid path connector of the accommodating component 511 through a plastic hose, and the second liquid inlet 722 is used for the cell suspension to enter. The third liquid inlet 720 is fluidly communicated with the liquid path connector of the accommodating component 521 through a plastic hose, and the third liquid inlet 720 is used for the biochemical reagents to enter. In this case, the air path connector of the accommodating component 510 is in fluid communication with the air pump outlet 302 of the air pump component 310, and the air path connector of the accommodating component 521 is in fluid communication with the air pump outlet 312 of the air pump component 320. The cell suspension and the biochemical reagents in the sample tubes of the accommodating component 510 and the accommodating component 511 can be driven by the same air pump component 310, as long as a branch is made on the pipeline. The waste liquid and collection liquid can be directly collected into PCR instrument, the tube of cell life support device, or culture dish.

In some embodiments, after collecting the droplets wrapped in oil phase, the method 1200 further comprises: S1230 incubating the droplets wrapped in oil phase.

The incubation treatment can be carried out in other devices (for example, other general-purpose PCR instruments, cell life support devices, etc.), and the incubation treatment can include dyeing, transfection, culture and other operations of cells.

In some embodiments, the cell processing chip further comprises a droplet sorting chip. The droplet sorting chip is configured to perform droplet sorting. The device further comprises a signal generating and processing member configured to apply a signal to a fluid in the cell processing chip to generate a response signal associated with the fluid, and configured to issue a control instruction in response to the response signal. The power supply is further configured to supply power to the signal generating and processing member and configured to apply a sorting signal to the cell processing chip in response to the control instruction. After incubating the droplets wrapped in oil phase, the method 1200 further comprises steps of S1240-1260.

S1240: fluidly communicating the accommodating member with the droplet sorting chip and fluidly communicating the accommodating member with the fluid driving member, coupling the signal generating and processing member with the droplet sorting chip, installing the droplet sorting chip on the bearing member, adding the incubated droplets wrapped in oil phase and oil phase liquid into the accommodating member.

For example, the droplet sorting chip is inserted into the chip fixing plate 711 and the fastening member 712 is tightened. The first liquid inlet 702 is fluidly communicated with the liquid path connector of the accommodating component 510 through the plastic hose, the second liquid inlet 722 is fluidly communicated with the liquid path connector of the accommodating component 511 through the plastic hose, and the liquid outlet 724 is fluidly communicated with the liquid path connector of the accommodating component 521 through the plastic hose. The air path connector of the accommodating component 510 is fluidly communicated with the air pump outlet 302 of the air pump component 310, the air path connector of the accommodating component 511 is fluidly communicated with the air pump outlet 312 of the air pump component 320, and the air pump inlets 303 and 313 are connected to the external air compressor or high-pressure gas cylinder. The chip fixing plate 711 connected with various connectors is inserted into the groove 202 of the chip base 201. The first optical transmission medium interface 703 and the second optical transmission medium interface 704 are connected to the first adapter 104 of the optical transmission medium adapter plate 100. The incubated droplet wrapped in oil phase is added into the sample tube of the accommodating component 511, and an oil phase liquid (droplet generation oil) is added to the sample tube of the accommodating component 510.

S1250: supplying power to the fluid driving member using the power supply, so as to drive the fluid in the accommodating member into the droplet sorting chip.

For example, the air compressor is started (or the switch of the high-pressure gas cylinder is turned on), the power supply is used to power the air pump controller. The ratio of air pressure for driving the sample tube of the accommodating component 510 and the sample tube of the accommodating component 511 is adjusted to control the liquid flow rate. The air pressure for driving the sample tube of the accommodating component 510 is increased firstly, so that oil phase liquid (droplet generation oil) first fills the droplet generation chip, and then the incubated droplet wrapped in oil phase is injected into the droplet sorting chip.

S1260: supplying power to the signal generating and processing member using the power supply to apply a signal to the fluid in the cell processing chip so as to generate a response signal associated with the fluid and issue a control instruction in response to the response signal, so that the power supply applies a sorting signal to the cell processing chip in response to the control instruction, thereby performing droplet sorting.

For example, the LED light source and PMT detector are started, the optical signal generated by the light source is used to illuminate the fluid, and the response signal of the fluid is collected and analyzed by the optical sensor 603. The third power supply 421 is started and the voltage is adjusted to 800V-1000V. The controller generates a sorting signal based on the PMT signal (response signal) received by the PMT detector, then controls the start and stop of the DC high voltage provided by the third power supply 421 and drives the high voltage electrode in the droplet sorting chip to discharge in response to the DC high voltage, so as to realize dielectrophoretic sorting and droplet sorting. For example, the analysis of the controller may be based on the wavelength difference between the signal transmitted by the light source and the signal received by the optical sensor. In some embodiments, the number of cells in the droplets can be analyzed based on the PMT signal, so as to realize the sorting of single-cell droplets.

For example, DC high voltage can be applied to the droplet where the target cell is located, and no voltage is applied to other droplets, so as to realize the sorting of different droplets. For example, a DC high voltage can be applied to other droplets while not applying a voltage to the droplets where the target cell is located, so as to realize the sorting of different droplets. For example, different voltages can be applied to the droplet where the target cell is located and other droplets respectively, so as to realize the sorting of different droplets.

The method provided by the embodiments of the present disclosure has similar advantages to the above device, and will not be repeated here.

As will be apparent to those skilled in the art, many different ways of performing the methods of these embodiments of the present disclosure are possible. For example, the order of steps can be changed, or some steps can be performed in parallel. In addition, other method steps can be inserted between steps. The inserted step may represent an improvement of a method such as that described herein, or may be independent of the method. In addition, a given step may not be fully completed before the next step begins. It should be understood that the features of different embodiments in the present disclosure can be combined with each other without contradiction.

Those skilled in the art can make various amendments and modifications to the present disclosure without departing from the spirit and scope of the present disclosure. Thus, if these amendments and modifications of the present disclosure fall within the scope of the claims of the present disclosure and their equivalents, the present disclosure is also intended to include these amendments and modifications.

1. A device for driving a cell processing chip, the cell processing chip being configured to process a cell, the device comprising:
a bearing member configured to carry the cell processing chip;
an accommodating member in fluid communication with the cell processing chip and including a space for accommodating a sample or reagent;
a fluid driving member configured to drive flow of fluid in the device and in the cell processing chip;
a signal generating and processing member configured to apply a signal to a fluid in the cell processing chip to generate a response signal associated with the fluid, and configured to issue a control instruction in response to the response signal; and
a power supply configured to supply power to the fluid driving member and the signal generating and processing member, and configured to apply a sorting signal to the cell processing chip in response to the control instruction.

2. The device according to claim 1, wherein the signal generating and processing member comprises:
a light source configured to provide an optical signal to the fluid in the cell processing chip through an optical transmission medium;
an optical sensor configured to receive a response signal of the fluid through the optical transmission medium; and
a processor configured to issue a control instruction based on a result of analyzing the response signal.

3. The device according to claim 2, wherein the light source is connected to a first position through the optical transmission medium, the optical sensor is connected to a second position through the optical transmission medium, the first position and the second position are in proximity to the cell processing chip and are respectively located on both sides of a flow channel of the cell processing chip, and a connecting line between the first position and the second position passes through the flow channel.

4. The device according to claim 3,
wherein the device further comprises a rack, which is provided with a space for accommodating functional components;
wherein the signal generating and processing member further comprises an optical transmission medium adapter plate detachably connected with the rack, and the optical transmission medium adapter plate is arranged between the light source and the first position or between the optical sensor and the second position, the optical transmission medium adapter plate comprises:
an optical transmission medium section comprising a first end and a second end opposite to the first end, wherein the first end is arranged at the first position when the optical transmission medium adapter plate is arranged between the light source and the first position, and the first end is arranged at the second position when the optical transmission medium adapter plate is arranged between the optical sensor and the second position,
wherein the optical transmission medium at the first end is exposed, and the first end is fixed relative to a microfluidic chip when the optical transmission medium adapter plate is in an installation position; and
wherein the second end is detachably connected to the light source when the first end is arranged at the first position, and the second end is detachably connected to the optical sensor when the first end is arranged at the second position.

5. The device according to claim 2, wherein the light source is connected to a first optical transmission medium interface in the cell processing chip through the optical transmission medium, and the optical sensor is connected to a second optical transmission medium interface in the cell processing chip through the optical transmission medium, the first optical transmission medium interface and the second optical transmission medium interface are respectively connected to a transmitter and a receiver located in the cell processing chip and arranged opposite to each other on both sides of the flow channel of the cell processing chip.

6. The device according to claim 5,
wherein the device further comprises a rack, which is provided with a space for accommodating functional components,
wherein the signal generating and processing member further comprises an optical transmission medium adapter plate detachably connected with the rack, and the optical transmission medium adapter plate is arranged between the light source and the first optical transmission medium interface or between the optical sensor and the second optical transmission medium interface, the optical transmission medium adapter plate comprises:
an optical transmission medium section comprising a first end and a second end opposite to the first end; and
a first adapter arranged at the first end and configured to detachably connect the first end and the first optical transmission medium interface when the optical transmission medium adapter plate is arranged between the light source and the first optical transmission medium interface, and detachably connect the first end and the second optical transmission medium interface when the optical transmission medium adapter plate is arranged between the optical sensor and the second optical transmission medium interface, and
wherein the second end is detachably connected to the light source when the optical transmission medium adapter plate is arranged between the light source and the first optical transmission medium interface, and the second end is detachably connected to the optical sensor when the optical transmission medium adapter plate is arranged between the optical sensor and the second optical transmission medium interface.

7. The device according to claim 4, wherein the optical transmission medium adapter plate further comprises:
a substrate detachably connected with the rack;
an optical transmission medium sleeve covering at least part of the optical transmission medium section; and
an adapter base connected with the optical transmission medium sleeve and the substrate, and the adapter base being provided with a through hole for the optical transmission medium to pass through.

8. The device according to claim 7, wherein the adapter base is provided with a recess, and the optical transmission medium sleeve extends at the recess so that an intersection line of the optical transmission medium sleeve and the recess forms an arc.

9. The device according to claim 1, wherein the fluid driving member comprises at least one air pump component, and each of the at least one air pump component comprises:
an air pump inlet in fluid communication with an air pressure device outside the device;
an air pump outlet configured to output high pressure gas; and
an air pump controller configured to control flow of the air pump inlet and the air pump outlet.

10. The device according to claim 9, wherein the accommodating member comprises at least one accommodating component, each of the at least one accommodating component comprises:
a sample tube;
an adapter arranged at one end of the sample tube and at least partially covering the sample tube;
an air path connector arranged on the adapter and configured to be in fluid communication with the air pump outlet; and
a liquid path connector arranged on the adapter and configured for liquid to enter and exit the sample tube.

11. The device according to claim 10,
wherein the at least one accommodating component comprises a first accommodating component, a second accommodating component and a third accommodating component, the cell processing chip comprises a first liquid inlet, a second liquid inlet and a liquid outlet, and
wherein liquid path connectors of the first accommodating component and the second accommodating component are in fluid communication with the first liquid inlet and the second liquid inlet respectively, and a liquid path connector of the third accommodating component is in fluid communication with the liquid outlet.

12. The device according to claim 11,
wherein the at least on air pump component comprises a first air pump component and a second air pump component, and
wherein air path connectors of the first accommodating component and the second accommodating component are in fluid communication with the air pump outlet of the first air pump component and the second air pump component respectively.

13. The device according to claim 4,
wherein the optical transmission medium adapter plate further comprises substrate positioning magnets distributed at four corners of a substrate, and the rack comprises rack positioning magnets, and
wherein the rack positioning magnets are magnetically combined with the substrate positioning magnets when the optical transmission medium adapter plate is in the installation position.

14. (canceled)

15. The device according to claim 1, wherein the bearing member comprises:
a chip base provided with a groove; and
a chip fixing plate configured to be embedded in the groove in an installation state, and the chip fixing plate comprising a notch,
wherein the cell processing chip is configured to be embedded in the notch in the installation state.

16. The device according to claim 15,
wherein the chip fixing plate comprises at least one fastening component, and
wherein when the at least one fastening component is in a fastening state, the at least one fastening component is configured to fasten the cell processing chip and the chip fixing plate to the chip base.

17. The device according to claim 4, further comprising:
a top plate; and
a bottom plate located on a side of the rack away from the top plate.

18. The device according to claim 1, wherein the power supply comprises a first power supply, a second power supply and a third power supply that are mutually independent, the first power supply is configured to supply power to the fluid driving member, the second power supply is configured to supply power to the signal generating and processing member, and the third power supply is configured to apply a sorting signal to the cell processing chip in response to the control instruction.

19. A method of driving a cell processing chip using the device according to claim 1, wherein the cell processing chip comprises a droplet generation chip, the method comprises:
fluidly communicating the accommodating member with the droplet generation chip and fluidly communicating the accommodating member with the fluid driving member, installing the droplet generation chip on the bearing member, adding an oil phase liquid and a cell suspension into the accommodating member; and
supplying power to the fluid driving member using the power supply, so as to drive the fluid in the accommodating member into the droplet generation chip, thereby generating droplets wrapped in oil phase, and collecting the droplets wrapped in oil phase.

20. The method according to claim 19, after collecting the droplets wrapped in oil phase, further comprising:
incubating the droplets wrapped in oil phase.

21. The method according to claim 20,
wherein the cell processing chip further comprises a droplet sorting chip,
wherein the device further comprises a signal generating and processing member configured to apply a signal to a fluid in the cell processing chip to generate a response signal associated with the fluid, and configured to issue a control instruction in response to the response signal,
wherein the power supply is further configured to supply power to the signal generating and processing member, and configured to apply a sorting signal to the cell processing chip in response to the control instruction, and
wherein after incubating the droplets wrapped in oil phase, the method further comprises:
fluidly communicating the accommodating member with the droplet sorting chip and fluidly communicating the accommodating member with the fluid driving member, coupling the signal generating and processing member with the droplet sorting chip, installing the droplet sorting chip on the bearing member, adding the incubated droplets wrapped in oil phase and oil phase liquid into the accommodating member;
supplying power to the fluid driving member using the power supply, so as to drive the fluid in the accommodating member into the droplet sorting chip; and
supplying power to the signal generating and processing member using the power supply to apply a signal to fluid in the cell processing chip so as to generate a response signal associated with the fluid and issue a control instruction in response to the response signal, so that the power supply applies a sorting signal to the cell processing chip in response to the control instruction, thereby performing droplet sorting.
